(19) United States
(12) Patent Application Publication
(10) Pub. No.: US 2023/0193163 A1
(43) Pub. Date: Jun. 22, 2023

(54) PSEUDOMONAS STUTZERI LIPASE AND USE THEREOF

(71) Applicant: Henkel AG & Co. KGaA, Duesseldorf (DE)

(72) Inventors: DANIELA HERBST, Duesseldorf (DE); NINA MUSSMANN, Willich (DE); SUSANNE WIELAND, Zons/Dormagen (DE); CLAUDIA LINDNER, Solingen (DE); SKANDER ELLEUCHE, Hamburg (DE); PHILIP BUSCH, Hamburg (DE); UTE LORENZ, Ellerbek (DE); GARABED ANTRANIKIAN, Seevetal-Hittfeld (DE)

(73) Assignee: Henkel AG & Co. KGaA, Duesseldorf (DE)

(21) Appl. No.: 16/606,354

(22) PCT Filed: Jun. 6, 2018
(86) PCT No.: PCT/EP2018/064850

§ 371 Date: Oct. 18, 2019

(30) Foreign Application Priority Data
Jun. 12, 2017 (DE) 10 2017 209 870.8

(51) Int. Cl. C11D 3/386; C11D 3/39; C11D 3/00; C11D 3/42

(52) U.S. Cl. CPC C11D 3/38627; C11D 3/0026; C11D 3/42; C11D 3/0047; C11D 3/3905

(57) ABSTRACT

The present disclosure is in the field of enzyme technology, in particular the lipolytic action of enzymes on fats and oils, as used, for example, in washing agents or cleaning agents. The present disclosure relates to an agent, in particular washing or cleaning agent, which contains a lipase as defined herein. Furthermore, the present disclosure relates to a method for the cleaning of textiles and the use of the agent as contemplated herein for the removal of soilings.

### CROSS-REFERENCE TO RELATED APPLICATION

This application is a U.S. National-Stage entry under 35 U.S.C. § 371 based on International Application No. PCT/EP2018/064850, filed Jun. 6, 2018, which was published under PCT Article 21(2) and which claims priority to German Application No. 10 2017 209 870.8, filed Jun. 12, 2017, which are all hereby incorporated in their entirety by reference.

### TECHNICAL FIELD

The present disclosure is in the field of enzyme technology, in particular the lipolytic action of enzymes on fats and oils, as used, for example, in washing agents or cleaning agents. The present disclosure relates to an agent, in particular washing or cleaning agent, which contains a lipase as defined herein. Furthermore, the present disclosure relates to a method for the cleaning of textiles and the use of the agent as contemplated herein for the removal of soilings.

### BACKGROUND

Lipases are among the most technically important enzymes. Their use for washing and cleaning agents is established industrially and they can be contained in modem, powerful washing and cleaning agents. Lipases are enzymes that catalyze the hydrolysis of ester bonds in lipid substrates, in particular in fats and oils, and thus belong to the group of esterases. Lipases are typically enzymes that can cleave a variety of substrates, for example, aliphatic, alicyclic, bicyclic and aromatic esters, thioesters and activated amines. Lipases are used to remove fat-containing soilings by catalyzing their hydrolysis (lipolysis). Lipases having broad substrate spectra are used in particular where inhomogeneous raw materials or substrate mixtures have to be transformed, for example, in washing agents and cleaning agents, since soiling can include differently structured fats and oils. The lipases used in the washing or cleaning agents known from the prior art are usually of microbial origin and are generally derived from bacteria or fungi, for example, the genera Bacillus, Pseudomonas, Acinetobacter, Micrococcus, Humicola, Trichoderma or Trichosporon. Lipases are usually produced according to biotechnological methods known per se by suitable microorganisms, for example, by transgenic expression hosts of the genera Bacillus or by filamentous fungi.

European patent application EP 443063, for example, discloses a lipase from Pseudomonas sp. ATCC 21808 provided for washing and cleaning agents. Japanese patent application JP 1225490 discloses a lipase from Rhizopus oryzae. In general, only selected lipases are suitable for use in liquid surfactant-containing preparations. Many lipases do not show sufficient catalytic performance or stability in such formulations. In particular, many lipases show thermal instability in washing processes which are generally performed at temperatures higher than about 20° C., which in turn leads to insufficient catalytic activity during the washing process. This problem is even more serious in phosphonate-containing liquid surfactant preparations, for example, due to the complex-forming properties of the phosphonates or due to unfavorable interactions between the phosphonate and the lipase.

Consequently, lipase and surfactant-containing liquid formulations of the prior art have the disadvantage in that they often do not have satisfactory lipolytic activity in the temperature ranges required by a washing process and therefore do not show optimal cleaning performance on lipase-sensitive soilings.

### BRIEF SUMMARY

Surprisingly, it has been found that a lipase from Pseudomonas stutzeri as described herein is active under washing process conditions and has good lipolytic properties. The sequence of the lipase identified herein has no significant sequence homologies to lipases heretofore used in washing or cleaning agents. It therefore opens up many possibilities to increase the genetic diversity of commercially used lipases and, if necessary, to change the performance spectrum through mutagenesis.

Therefore, in a first aspect, the present disclosure is directed to an agent, preferably a washing or cleaning agent, that contains at least one lipase that has at least about 65% sequence identity with the amino acid sequence specified in SEQ ID NO:1 over its entire length.

In a further aspect, the present disclosure is directed to methods for the cleaning of textiles, exemplified in that an agent as contemplated herein is applied in at least one method step.

In yet another aspect, the present disclosure is further directed to the use of an agent as described herein, preferably a washing or cleaning agent, more preferably a liquid washing agent, for the removal of (fat-containing) soilings.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The lipases as contemplated herein have enzymatic activity, that is, they are capable of the hydrolysis of fats and oils, in particular in a washing or cleaning agent. A lipase as contemplated herein is therefore an enzyme which catalyzes the hydrolysis of ester bonds in lipid substrates and thereby is able to cleave fats or oils. Furthermore, a lipase as contemplated herein is preferably a mature lipase, that is, the catalytically active molecule without signal and/or propeptide(s). Unless otherwise stated, the sequences given refer to respectively mature (processed) enzymes.

In various preferred embodiments of the present disclosure, the lipase is a lipase having at least about 70% sequence identity with the amino acid sequence specified in SEQ ID NO: 1 over its entire length. In further preferred embodiments, the lipase contained in the agent as contemplated herein comprises, essentially consists of or consists of the amino acid sequence specified in SEQ ID NO:1. In various embodiments, , the present disclosure also encompasses lipases derived from the amino acid sequence according to SEQ ID NO:1, for example, by employing mutagenesis. In various further embodiments, the present disclosure also comprises lipases which are obtainable by the expression of a nucleotide sequence which codes for a protein according to SEQ ID NO:1. In one aspect, , the present disclosure also encompasses nucleotide sequences which are identical to the nucleotide sequence which codes for the protein according to SEQ ID NO: 1 over the total length thereof to at least about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88 %, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4% or about 99.6%, provided that the native sequence coding for the lipase from Pseudomonas stutzeri is excluded.

In various embodiments as contemplated herein, the lipase comprises an amino acid sequence which is identical to the amino acid sequence specified in SEQ ID NO: 1 over its total length to at least about 66%, about 67%, about 68%, about 69%, about 70%, about 71%, about 72%, about 73%, about 74%, about 75%, 7 about 6%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.8%, about 99.0%, about 99.2%, about 99.4% or about 99.6% or includes such a sequence.

In various embodiments as contemplated herein, the agent is exemplified in that
(a) the lipase is obtainable from a lipase as defined above as a starting molecule by single or multiple conservative amino acid substitution; and or
(b) the lipase is obtainable from a lipase as defined above as a starting molecule by fragmentation, deletion, insertion or substitution mutagenesis and comprises an amino acid sequence which matches over a length of at least about 182, about 190, about 200, about 210, about 220, about 230, about 240, about 245, about 250, about 260, about 270, about 271, about 272, about 273, about 274, about 275, about 276, about 277, about 278, about 279 or about 280 contiguous amino acids with the starting molecule.

The agents as contemplated herein preferably contain the lipase in an amount of from about 0.00001-1% by weight, more preferably in an amount of from about 0.0001-0.5% by weight, particularly preferably in an amount of from about 0.001-0.1% by weight, each based on the active protein.

The identity of nucleic acid or amino acid sequences is determined by a sequence comparison. This sequence comparison is based on the BLAST algorithm established and commonly used in the prior art (see, for example, Altschul, S.F., Gish, W., Miller, W., Myers, E.W. & Lipman, D.J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, pp. 3389-3402) and in principle occurs by assigning similar sequences of nucleotides or amino acids in the nucleic acid or amino acid sequences to one another. A tabular assignment of the respective positions is referred to as alignment. A further algorithm available in the prior art is the FASTA algorithm. Sequence comparisons (alignments), in particular multiple sequence comparisons, are created with computer programs. For example, the Clustal series are frequently used (see, for example, Chenna et al. (2003): Multiple sequence alignment with the Clustal series of programs. Nucleic Acid Research 31, 3497-3500), T-Coffee (see, for example, Notredame et al., (2000): T-Coffee: A novel method for multiple sequence alignments. J. Mol. Biol. 302, 205-217) or programs based on these programs or algorithms. Also possible are sequence comparisons (alignments) using the computer program Vector NTI® Suite 10.3 (Invitrogen Corporation, 1600 Faraday Avenue, Carlsbad, California, USA) with the default parameters whose AlignX module for sequence comparisons is based on ClustalW.

Such a comparison also allows a statement about the similarity of the sequences compared to each other. It is usually given in percent identity, that is, the proportion of identical nucleotides or amino acid residues at the same or in positions corresponding an alignment with each other. The broader concept of homology involves conserved amino acid substitutions in amino acid sequences, that is, amino acids having similar chemical activity, as these usually perform similar chemical activities within the protein. Therefore, the similarity of the sequences compared can also be stated as percent homology or percent similarity. Identity and/or homology specifications can be made about whole polypeptides or genes or only over individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It can therefore be useful to relate sequence matches only to individual, possibly small regions. Unless otherwise indicated, identity or homology specification in the present application, however, refers to the total length of the respectively specified nucleic acid or amino acid sequence.

In various embodiments, the lipase comprises an amino acid sequence which is homologous to the amino acid sequence specified in SEQ ID NO: 1 over its total length at least about 66%, about 67%, about 68%, about 69%, about 70%, about 71%, about 72%, about 73%, about 74 %, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96, about 5%, about 97%, about 97.5%, about 98%, about 98.5%, about 98.8%, about 99.0%, about 99.2%, about 99.4% or about 99.6%.

In a further embodiment,, the lipase is characterized in that its lipolytic performance is not significantly reduced compared to that of a lipase comprising an amino acid sequence corresponding to the amino acid sequences specified in SEQ ID NO:1, that is, at least about 70%, about 75%, about 80%, about 85%, about 90%, about 95% of the reference performance. The lipolytic performance can be determined in a washing system containing a washing agent in a dosage between about 4.5 and about 7.0 grams per liter of wash solution and the lipase, wherein the lipases to be compared in concentration (based on active protein) are used and the lipolytic performance is determined as described herein. For example, the washing process can be carried out for about 60 minutes at a temperature of about 60° C. and the water have a water hardness between about 15.5 and about 16.5° (German hardness). The concentration of the lipase in the washing agent determined for this washing system is from about 0.00001 to about 1% by weight, preferably from about 0.0001 to about 0.5% by weight, particularly preferably from about 0.001 to about 0.1% by weight based on active, purified protein.

A preferred liquid washing agent for such a washing system is composed as follows (all specifications in weight percent): anionic surfactant from about 5-7%, builder (for example, citric acid and phosphonates) from about 0-1%, caustic soda from about 0-1%, palm kernel fatty acid from about 0-1%, glycerol from about 0-1%, sodium chloride from about 1-3%, boric acid from about 0-1%, further additives (preservative, defoamer, opt. brightener, dye, perfume) from about 0-1% and balance demineralized water. The dosage of the liquid washing agent is preferably between about 4.5 and about 6.0 grams per liter wash solution, for example, from about 4.7, 4.9 or about 5.9 grams per liter wash solution. Preference is given to washing in a pH range between about pH 8 and about pH 10.5, preferably between about pH 8 and about pH 9.

In the context of the present disclosure, the determination of the lipolytic performance is carried out at about 40° C. using a liquid washing agent as indicated above, wherein the washing process is preferably carried out for about 60 minutes.

The degree of whiteness, that is, the brightening of the soiling, as a measure of the cleaning performance, is determined by optical measuring methods, preferably photometrically. A suitable device for this purpose is, for example, the spectrometer Minolta CM508d. Usually, the devices used for the measurement are previously calibrated with a white standard, preferably a supplied white standard.

The activity-like use of the respective lipase ensures that, even with a possible divergence of ratio of active substance to total protein (the values of the specific activity), the respective enzymatic properties, thus, for example, the cleaning performance of certain soilings, are compared. In general, a low specific activity can be compensated by adding a larger amount of protein.

The lipase activity can otherwise also be determined in the usual manner, preferably as described in Bruno Stellmach, “Bestimmungsmethoden Enzyme fur Pharmazie, Lebensmittelchemie, Technik, Biochemie, Biologie, Medizin” (Steinkopff Verlag Darmstadt, 1988, p. 172ff). In this case, lipase-containing samples are added to an olive oil emulsion in emulsifier-containing water and incubated at about 30° C. and about pH 9.0. Fatty acids are thereby released. These are titrated using a auto-titrator over about 20 minutes continuously with about 0.01 N sodium hydroxide solution, so that the pH remains constant (“pH-stat titration”). Based on the sodium hydroxide consumption, the determination of the lipase activity is carried out by reference to a reference lipase sample.

An alternative test for determining the lipolytic activity of the lipases as contemplated herein is an optical measuring method, preferably a photometric method. The appropriate test for this involves the lipase-dependent cleavage of the substrate para-nitrophenol butyrate (pNP-butyrate). This is cleaved by the lipase into para-nitrophenolate and butyrate. The presence of para-nitrophenolate can be determined using a photometer, for example, the Tecan Sunrise device and the XFLUOR software, at about 405 nm and thus enables a conclusion about the enzymatic activity of the lipase.

Proteins can be summarized into groups of immunologically related proteins via the reaction with an antiserum or a specific antibody. The members of such a group are characterized by having the same antigenic determinant recognized by an antibody. They are therefore structurally so similar to each other that they are recognized by an antiserum or specific antibodies. Lipases therefore form a further subject of the present disclosure, which lipases have at least one and increasingly preferably two, three or four matching antigenic determinants with a lipase used in an agent as contemplated herein. Due to their immunological similarity, such lipases are structurally so similar to the lipases used in the agents as contemplated herein that a similar function can also be assumed.

Further lipases used in the agents as contemplated herein can, in comparison to the lipase described in SEQ ID NO:1, have further amino acid changes, in particular amino acid substitutions, insertions or deletions. Such lipases are, for example, further developed by targeted genetic modification, that is, by mutagenesis methods, and optimized for specific applications or with regard to specific properties (for example, with regard to their catalytic activity, stability, etc.). Furthermore, nucleic acids which code the used lipases can be introduced into recombination approaches and thus used to generate completely novel lipases or other polypeptides.

The goal is to introduce into the known molecules targeted mutations such as substitutions, insertions or deletions, for example, to improve the cleaning performance of enzymes as contemplated herein. For this purpose, in particular, the surface charges and/or the isoelectric point of the molecules and thereby their interactions with the substrate can be changed. Thus, for example, the net charge of the enzymes can be changed in order to influence the substrate binding, in particular for use in washing and cleaning agents. Alternatively or additionally, one or more corresponding mutations can increase the stability of the lipase and thereby improve its cleaning performance. Advantageous properties of individual mutations, for example, individual substitutions, can be complementary. A lipase which has already been optimized with regard to certain properties, for example with respect to its activity, can therefore be further developed within the scope as contemplated herein.

A further object of the present disclosure is therefore an agent containing a lipase, which is obtainable from a lipase as described above as the starting molecule by one or more conservative amino acid substitution. The term “conservative amino acid substitution” means the substitution of one amino acid residue for another amino acid residue, wherein this substitution does not lead to a change in polarity or charge at the position of the exchanged amino acid, for example, the replacement of a nonpolar amino acid residue for another nonpolar amino acid residue. Conservative amino acid substitutions within the scope as contemplated herein include, for example: G=A=S, I=V=L=M, D=E, N=Q, K=R, Y=F, S=T, G=A=I=V=L=M=Y=F=W=P=S=T. The homology of the thus modified lipases to the lipase having SEQ ID NO:1 is preferably as defined above.

Alternatively or additionally, the lipase is obtainable from a lipase contained in an agent as contemplated herein as a starting molecule by fragmentation, deletion, insertion or substitution mutagenesis and comprises an amino acid sequence that matches the starting molecule over a length of at least about 182, about 190, about 200, about 210, about 220, about 230, about 240, about 245, about 250, about 260, about 270, about 271, about 272, about 273, about 274, about 275, about 276, about 277, about 278, about 279 or about 280 contiguous amino acids.

Thus, for example, it is possible to delete individual amino acids at the termini or in the loops of the enzyme without thereby losing or reducing the hydrolytic activity. Furthermore, such fragmentation, deletion, insertion or substitution mutagenesis can also reduce, for example, the allergenicity of the enzymes concerned and thus improve their overall applicability. Advantageously, the enzymes retain their hydrolytic activity even after mutagenesis, that is, their hydrolytic activity is at least equal to that of the starting enzyme, that is, in a preferred embodiment, the hydrolytic activity is at least about 80, preferably at least about 90% of the activity of the starting enzyme. Substitutions can also show further beneficial effects. Both single and multiple contiguous amino acids can be substituted for other amino acids.

In various embodiments, in addition to the sequence specified in SEQ ID NO: 1, the lipase can have one or more further amino acids N— or C-terminal. In certain embodiments, such N-terminal peptides can be the naturally occurring signal peptides for the lipase or even a single methionine radical.

An object of the present disclosure is an agent that contains a lipase as defined herein. The agent is preferably a washing or cleaning agent.

All percent specifications made in connection with the compositions/agents described herein are, unless explicitly stated otherwise, refer to % by weight, in each case based on the respective mixture/agent.

In the context of the present disclosure fatty acids or fatty alcohols or their derivatives, unless otherwise stated, stand for branched or unbranched carboxylic acids or alcohols or their derivatives having preferably 6 to 22 carbon atoms. In particular, the oxo alcohols or their derivatives, which are obtainable, for example, by the Roelen oxo synthesis, can also be used correspondingly.

Whenever alkaline earth metals are referred to below as counterions for monovalent anions, this means that the alkaline earth metal is naturally present only in half the amount of substance as the anion, as sufficient to balance the charge.

This disclosure includes all conceivable types of washing agents or cleaning agents, both concentrates and applied agents, for use on a commercial scale, in the washing machine or in hand washing. These include washing agents for textiles, carpets, or natural fibers, for which the term washing agent is used. The washing and cleaning agents in the context of the present disclosure also include washing aids, which are metered into the actual washing agent in manual or automatic textile washing, in order to achieve a further effect. Furthermore, laundry washing and cleaning agents in the context of the present disclosure also include textile pre-treatment and post-treatment agents, that is, those agents with which the item to be washed is brought into contact before the actual washing, for example, to dissolve stubborn soiling, and also such agents, which in a step downstream the actual textile washing give the laundry further desirable properties such as comfortable grip, crease resistance or low static charge. Fabric softeners are included in the just named agents.

The washing or cleaning agents as contemplated herein, which can be in the form of powdered solids, in redensified particle form, as homogeneous solutions, gels or suspensions, can contain, in addition to the above-described lipase, all known ingredients customary in such agents, wherein preference is given to at least one further ingredient is present in the agent. The agents as contemplated herein can in particular contain surfactants, builders, bleaching agents, in particular peroxygen compounds, or bleach activators. Furthermore, they can contain water-miscible organic solvents, further enzymes, sequestering agents, electrolytes, pH regulators and/or further auxiliaries, such as optical brighteners, grayness inhibitors, foam regulators and dyes and perfumes, and combinations thereof.

In particular, a combination of the agent as contemplated herein with one or more further ingredient(s) is advantageous, since such agent has an improved cleaning performance by resulting synergisms in preferred embodiments of the present disclosure. In particular, such a synergism can be achieved by combining the agent as contemplated herein with a surfactant and/or a builder and/or a peroxygen compound and/or a bleach activator.

Advantageous ingredients of agents as contemplated herein are disclosed in international patent application WO2009/121725, starting on page 5, penultimate paragraph, and ending on page 13 after the second paragraph. This disclosure is expressly incorporated herein by reference and the disclosure therein is included in the present patent application.

These and other aspects, features, and advantages of the present disclosure become apparent to those skilled in the art from a study of the following detailed description and claims. Any feature of one aspect of the present disclosure can be used in any other aspect of the present disclosure. Furthermore, it is to be understood that the examples contained herein are intended to describe and illustrate the present disclosure, but not to limit it, and in particular that the present disclosure is not limited to these examples. All percentage specifications are by percent weight unless otherwise specified, based on the total weight of the composition. Numeric ranges indicated in the format “from x to y” include the named values. When multiple preferred numeric ranges are specified in this format, it is understood that all ranges resulting from the combination of the various endpoints are also included.

In addition to the lipase, the agents as contemplated herein preferably also contain at least one compound from the class of surfactants, in particular selected from anionic and nonionic, but also cationic, zwitterionic or amphoteric surfactants.

Suitable surfactants are, for example, anionic surfactants of the formula (I)

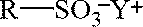

In this formula (I), R stands for a linear or branched unsubstituted alkylaryl radical. Y stands for a monovalent cation or the n-th part of an n-valent cation, the alkali metal ions being preferred, and Na⁺ or K⁺ being preferred, wherein Na⁺ is extremely preferred. Further cations Y⁺ can be selected from NHa+, %2 Zn²⁺, %2 Mg²⁺, %2 Ca²⁺, %2 Mn²⁺, and mixtures thereof.

“Alkylaryl” as used herein refers to organic radicals including an alkyl radical and an aromatic radical. Typical examples of such radicals include, but are not limited to, alkylbenzene radicals such as benzyl, butylbenzene radicals, nonylbenzene radicals, decylbenzene radicals, undecylbenzene radicals, dodecylbenzene radicals, tridecylbenzene radicals, and the like.

In various embodiments, such surfactants are selected from linear or branched alkylbenzenesulfonates of the formula A-1

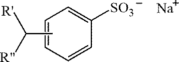

in which R′ and R″ together contain 9 to 19, preferably 11 to 15 and in particular 11 to 13 C atoms. A particularly preferred representative can be described by the formula A—la:

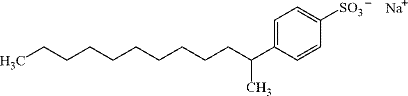

In various embodiments, the compound of formula (I) is preferably the sodium salt of a linear alkyl benzene sulfonate.

In agents as contemplated herein, the at least one compound from the class of anionic surfactants of the formula (I) can be present in an amount of from about 0.001 to about 30% by weight, preferably from about 0.001-10% by weight, more preferably from about 2-6% by weight. still more preferably from about 3-5% by weight, in the washing or cleaning agent, each based on the total weight of the cleaning agent.

In various embodiments, the agents as contemplated herein preferably contain at least one anionic surfactant of the formula

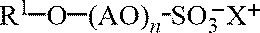

In this formula (II), R¹ stands for a linear or branched, substituted or unsubstituted alkyl, aryl or alkylaryl radical, preferably a linear, unsubstituted alkyl radical, more preferably a fatty alcohol radical. Preferred radicals R¹ are selected from decyl, undecyl, dodecyl, tridecyl, tetradecyl, pentadecyl, hexadecyl, heptadecyl, octadecyl, nonadecyl, eicosyl radicals and mixtures thereof, wherein the representatives with an even number of carbon atoms are preferred. Particularly preferred radicals R¹ are derived from C₁₂-C₁₈ fatty alcohols, for example, coconut fatty alcohol, tallow fatty alcohol, lauryl, myristyl, cetyl or stearyl alcohol or C₁₀-C₂₀ oxo alcohols.

AO stands for an ethylene oxide (EO) or propylene oxide (PO) grouping, preferably an ethylene oxide grouping. The index n is an integer from about 1 to about 50, preferably from about 1 to about 20 and in particular from about 1 to about 10. Most preferably, n stands for the numbers 2, 3, 4, 5, 6, 7 or 8. X stands for a monovalent cation or the n-th part of an n-valent cation, the alkali metal ions being preferred, and Na⁺ or K⁺ being preferred, wherein Na⁺ is extremely preferred. Further cations X+ can be selected from NH₄⁺, ½ Zn²⁺, ½ Mg²⁺, ½ Ca²⁺, ½ Mn²⁺, and mixtures thereof.

In summary, agents in various embodiments thus contain at least one anionic surfactant selected from fatty alcohol ether sulfates of the formula A-2

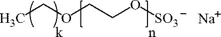

with k = 11 to 19, n = 2, 3, 4, 5, 6, 7 or 8. Very particularly preferred representatives are Na—C₁₂—₁₄ fatty alcohol sulfates 2 EO (k = 11-13, n = 2 in formula A-2).

In various embodiments, the cleaning agent contains the at least one anionic surfactant of the formula (II) in an amount of from about 2-10% by weight, preferably from about 3-8% by weight, based on the total weight of the cleaning agent.

Further usable anionic surfactants are the alkyl sulfates of the formula

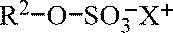

In this formula (III), R² stands for a linear or branched, substituted or unsubstituted alkyl radical, preferably a linear, unsubstituted alkyl radical, more preferably a fatty alcohol radical. Preferred radicals R² are selected from decyl, undecyl, dodecyl, tridecyl, tetradecyl, pentadecyl, hexadecyl, heptadecyl, octadecyl, nonadecyl, eicosyl radicals and mixtures thereof, wherein the representatives with an even number of carbon atoms are preferred. Particularly preferred radicals R² are derived from C₁₂-C₁₈ fatty alcohols, for example, coconut fatty alcohol, tallow fatty alcohol, lauryl, myristyl, cetyl or stearyl alcohol or C₁₀-C₂₀ oxo alcohols. Y stands for a monovalent cation or the n-th part of an n-valent cation, the alkali metal ions being preferred, and Na⁺ or K⁺ being preferred, wherein Na⁺ is extremely preferred. Further cations Y+ can be selected from NH₄⁺, %2 Zn²⁺, %2 Mg²⁺, %2 Ca²⁺, %2 Mn²⁺, and mixtures thereof.

In various embodiments, these surfactants are selected from fatty alcohol sulfates of formula A-3

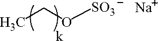

with k = 11 to 19. Very particularly preferred representatives are Na—C₁₂—₁₄ fatty alcohol sulfates (k = 11-13 in formula A-3).

In various embodiments, the agent can contain, in addition to the anionic surfactants described above, in particular those of the formulas (I)-(III), or alternatively thereto at least one other surfactant. In particular, further anionic surfactants, nonionic surfactants and mixtures thereof, but also cationic, zwitterionic and amphoteric surfactants are regarded as suitable alternative or additional surfactants.

In various embodiments, the agents comprise at least one nonionic surfactant, in particular at least one fatty alcohol alkoxylate.

Suitable nonionic surfactants are those of the formula

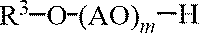

in which
R³ stands for a linear or branched, substituted or unsubstituted alkyl radical,
AO stands for an ethylene oxide (EO) or propylene oxide (PO) grouping,
m stands for an integer from about 1 to about 50.

In the formula (IV) given above, R³ stands for a linear or branched, substituted or unsubstituted alkyl radical, preferably a linear, unsubstituted alkyl radical, more preferably a fatty alcohol radical. Preferred radicals R² are selected from decyl, undecyl, dodecyl, tridecyl, tetradecyl, pentadecyl, hexadecyl, heptadecyl, octadecyl, nonadecyl, eicosyl radicals and mixtures thereof, wherein the representatives with an even number of carbon atoms are preferred. Particularly preferred radicals R³ are derived from C₁₂-C₁₈ fatty alcohols, for example, coconut fatty alcohol, tallow fatty alcohol, lauryl, myristyl, cetyl or stearyl alcohol or C₁₀-C₂₀ oxo alcohols.

AO stands for an ethylene oxide (EO) or propylene oxide (PO) grouping, preferably an ethylene oxide grouping. The index n stands for an integer from about 1 to about 50, preferably from about 1 to about 20 and in particular from about 2 to about 10. Most preferably, n stands for the numbers 2, 3, 4, 5, 6, 7 or 8.

In summary, preferably used fatty alcohol alkoxylates are compounds of the formula

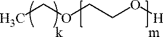

with k = 11 to 19, m = 2, 3, 4, 5, 6, 7 or 8. Very particularly preferred representatives are C₁₂-₁₈ fatty alcohols with 7 EO (k = 11-17, m = 7 in formula (V)).

Further nonionic surfactants which can be present in the described agents in the context of the present disclosure include, but are not limited to, alkyl glycosides, alkoxylated fatty acid alkyl esters, amine oxides, fatty acid alkanolamides, hydroxy mixed ethers, sorbitan fatty acid esters, polyhydroxy fatty acid amides, and alkoxylated alcohols.

Suitable amphoteric surfactants are, for example, betaines of the formula (Rⁱⁱⁱ)(R^{iV})(R^{V})N⁺CH₂COO⁻, in which Rⁱⁱⁱ is an alkyl radical optionally interrupted by hetero atoms or heteroatom groups, the alkyl radical having 8 to 25, preferably 10 to 21 carbon atoms and R′° and R^{v} are identical or different alkyl radicals having 1 to 3 carbon atoms, in particular Cio-Cis alkyl dimethylcarboxymethyl betaine and C₁₁-C₁₇ alkylamidopropyl-dimethylcarboxymethyl betaine.

Suitable cationic surfactants are, among other things, the quaternary ammonium compounds of the formula (R^{vi})(R^{vii})(R^{viii})(R^{ix})N⁺ X⁻, in which R^{vi} to R^{ix} stand for four identical or different, in particular two long and two short-chain, alkyl radicals and X⁻ for an anion, in particular a halide ion, for example, didecyl dimethyl ammonium chloride, alkyl benzyl dodecyl ammonium chloride and mixtures thereof. Further suitable cationic surfactants are the quaternary surface-active compounds, in particular having a sulfonium, phosphonium, iodonium or arsonium group, which are also known as antimicrobial active ingredients. Through the use of quaternary surface-active compounds having antimicrobial action, the agent can be designed with an antimicrobial effect or its possibly existing antimicrobial effect due to other ingredients can be improved.

In various embodiments, the total amount of the surfactants based on the weight of the composition is from about 2 to about 30% by weight, preferably from about 5 to about 25% by weight, more preferably from about 10 to about 20% by weight, most preferably from about 14 to about 18% by weight, wherein the (linear) alkylbenzenesulfonates are present at most in an amount of from about 0.001 to about 30% by weight, preferably from about 0.001-10% by weight, more preferably from about 2-6% by weight, more preferably from about 3-5% by weight, based on the weight of the agent.

Washing or cleaning agents as contemplated herein can contain further enzymes in addition to the lipase. These can be hydrolytic enzymes or other enzymes in a concentration practical for the effectiveness of the agent. One embodiment as contemplated herein thus represents agents comprising one or more enzymes. All enzymes which can display a catalytic activity in the agent as contemplated herein can be used as an enzyme, in particular a protease, amylase, cellulase, hemicellulase, mannanase, tannase, xylanase, xanthanase, xyloglucanase, P-glucosidase, pectinase, carrageenase, perhydrolase, oxidase, oxidoreductase, cutinase or other lipases, and mixtures thereof. Enzymes are advantageously present in the agent in each case in an amount of 1 × 10⁻⁸ to 5% by weight based on active protein. More preferably, each enzyme is present in an amount of 1 × 10⁻⁷ - 3% by weight, from about 0.00001 to about 1% by weight, from about 0.00005 to about 0.5% by weight, from about 0.0001 to about 0.1% by weight and particularly preferably from about 0.0001 to about 0.05% by weight in agents as contemplated herein, based on active protein. Particularly preferably, the enzymes show synergistic cleaning performance against certain soilings or stains, that is, the enzymes contained in the agent composition mutually support each other in their cleaning performance. Synergistic effects can occur not only between different enzymes, but also between one or more enzymes and further ingredients of the agent as contemplated herein.

The further lipase(s) is/are preferably an a-amylase. The hemicellulase is preferably a β-glucanase, a pectinase, a pullulanase and/or a mannanase. The cellulase is preferably a cellulase mixture or a one-component cellulase, preferably or predominantly an endoglucanase and/or a cellobiohydrolase. The oxidoreductase is preferably an oxidase, in particular a choline oxidase, or a perhydrolase.

The proteases used are preferably alkaline serine proteases. They act as nonspecific endopeptidases, that is, they hydrolyze any acid amide bonds that are located inside peptides or proteins and thereby cause degradation of proteinaceous soilings on the items to be cleaned. Their pH optimum is usually in the clearly alkaline range.

The protein concentration can be determined with the help of known methods, for example, the BCA method (bicinchoninic acid, 2,2′-biquinolyl-,4′-dicarboxylic acid) or the biuret method. The determination of the active protein concentration is carried out in this regard via a titration of the active sites using a suitable irreversible inhibitor (for proteases, for example, phenylmethylsulfonyl fluoride (PMSF)) and determination of the residual activity (compare M. Bender et al., J. Am. Chem. Soc. 88, 24 (1966), p. 5890-5913).

The enzymes to be used can further be formulated together with adjuncts, for example, from fermentation, in the cleaning agents described herein. In liquid formulations, the enzymes are preferably used as enzyme liquid formulation(s).

The enzymes are usually not provided in the form of the pure protein, but rather in the form of stabilized, storable and transportable preparations. Such prefabricated preparations include, for example, the solid preparations obtained by granulation, extrusion or lyophilization or, in particular in the case of liquid or gel-form agents, solutions of the enzymes, advantageously as concentrated as possible, low in water and/or added with stabilizers or further auxiliaries.

Alternatively, the enzymes can be encapsulated for both the solid and liquid dosage forms, for example, by spray-drying or extruding the enzyme solution together with a preferably natural polymer or in the form of capsules, for example, those in which the enzymes are enclosed as in a solidified gel or in those of the core-shell type, in which an enzyme-containing core is coated with a water, air and/or chemical impermeable protective layer. Additional active ingredients, for example, stabilizers, emulsifiers, pigments, bleaches or dyes, can additionally be applied in deposited layers. Such capsules are applied by methods known per se, for example, by shaking or rolling granulation or in fluid-bed processes. Advantageously, such granules are low in dust and storage stable due to the coating, for example, by applying polymeric film-forming agents.

Furthermore, it is possible to formulate two or more enzymes together so that a single granule has a plurality of enzyme activities.

The agent as contemplated herein can have one or more enzyme stabilizers in various embodiments. Therefore, the agent as contemplated herein can further contain an enzyme stabilizer, for example, selected from the group consisting of sodium formate, sodium sulfate, lower aliphatic alcohols and boric acid and their esters and salts. Of course, two or more of these compounds can be used in combination. The salts of the compounds mentioned can also be used in the form of hydrates, such as, sodium sulfate decahydrate.

The term “lower aliphatic alcohols”, as used herein, includes monoalcohols, diols, and higher alcohols having up to 6 carbon atoms. Polyols, for example, glycerol, (mono)ethylene glycol, (mono)propylene glycol or sorbitol can be mentioned as belonging to the group of lower aliphatic alcohols in this context, without the present disclosure being restricted thereto.

In addition to the at least one enzyme stabilizer selected from the above group, an agent as contemplated herein can also contain at least one further stabilizer. Such stabilizers are known in the prior art.

Reversible protease inhibitors protect the enzymes contained in a washing or cleaning agent from proteolytic degradation by reversibly inhibiting the enzymatic activity of the proteases contained in the agent. Boronic acids or their salts or esters are frequently used as reversible protease inhibitors benzamidine hydrochloride, including primarily derivatives having aromatic groups, such as ortho-, meta- or para-substituted phenylboronic acids, in particular 4-formylphenyl-boronic acid, or the salts or esters of said compounds. Also, peptide aldehydes, that is, oligopeptides having a reduced C-terminus, in particular those of from about 2 to about 50 monomers, are used for this purpose. The peptidic reversible protease inhibitors include, among others, ovomucoid and leupeptin.

Further enzyme stabilizers are amino alcohols such as mono-, di-, triethanol-and -propanolamine and mixtures thereof, aliphatic carboxylic acids up to C₁₂, such as succinic acid, other dicarboxylic acids or salts of said acids. End-capped fatty acid amide alkoxylates are also suitable for this purpose. Some organic acids used as builders are also able to stabilize an enzyme. Calcium and/or magnesium salts, such as calcium acetate, are also used for this purpose.

Polyamide oligomers or polymeric compounds such as lignin, water-soluble vinyl copolymers or cellulose ethers, acrylic polymers and/or polyamides stabilize the enzyme preparation, among other things, against physical influences or pH fluctuations. Polymers that contain polyamine N-oxide act simultaneously as enzyme stabilizers and as dye transfer inhibitors. Other polymeric stabilizers are linear C₈-C_{I8} polyoxyalkylenes. Alkylpolyglycosides can also stabilize the enzymatic components of the agent as contemplated herein and, preferably, are capable of additionally increasing their performance. Crosslinked N-containing compounds preferably fulfill a dual function as soil release agents and as enzyme stabilizers. Hydrophobic, nonionic polymer stabilizes, in particular, an optionally contained cellulase.

Reducing agents and antioxidants increase the stability of the enzymes to oxidative degradation; for example, sulfur-containing reducing agents are common for this purpose, for example, sodium sulfite and reducing sugars.

In one embodiment, the agents according to the present disclosure are liquid and contain water as the main solvent, that is, they are aqueous agents. The water content of the aqueous agent as contemplated herein is usually from about 15 to about 70% by weight, preferably from about 20 to about 60% by weight. In various embodiments, the water content is more than about 5% by weight, preferably more than about 15% by weight and particularly preferably more than about 50% by weight, each based on the total amount of agent.

In addition, nonaqueous solvents can be added to the agent. Suitable non-aqueous solvents include mono- or polyhydric alcohols, alkanolamines or glycol ethers, provided that they are miscible with water in the specified concentration range. Preferably, the solvents are selected from ethanol, n-propanol, i-propanol, butanols, glycol, propanediol, butanediol, methylpropanediol, glycerol, diglycol, propyldiglycol, butyldiglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol monomethyl ether, dipropylene glycol monoethyl ether, methoxytriglycol, ethoxytriglycol, butoxytriglycol, 1-butoxyethoxy-2-propanol, 3-methyl-3-methoxybutanol, propylene glycol t-butyl ether, di-n-octyl ether and mixtures of these solvents.

The one or more non-aqueous solvents is/are usually contained in an amount of from about 0.1 to about 10% by weight, preferably from about 1 to about 8% by weight, based on the total composition.

In addition to the components mentioned so far, the agents as contemplated herein can contain further ingredients which further improve the technical performance and/or aesthetic properties of the cleaning agent. These include, for example, additives for improving the flow and drying behavior, for adjusting the viscosity and/or for stabilization, and other cleaning agents and additives customary in cleaning agents, such as UV stabilizers, perfume, pearlescing agents, dyes, corrosion inhibitors, preservatives, bittering substances, organic salts, disinfectants, structuring polymers, defoamers, encapsulated ingredients (for example, encapsulated perfume), pH adjusters and additives to nourish skin or improve feel.

An agent as contemplated herein, in particular washing or cleaning agent, can contain at least one water-soluble and/or water-insoluble, organic and/or inorganic builder.

Among the builders which can generally be used are, in particular, the aminocarboxylic acids and their salts, zeolites, silicates, carbonates, organic (co)builders and, where there are no ecological prejudices against their use, also the phosphates. Preferably, however, the agents are phosphate-free.

The water-soluble organic builder substances include polycarboxylic acids, in particular citric acid and sugar acids, monomeric and polymeric aminopolycarboxylic acids, in particular methylglycinediacetic acid, nitrilotriacetic acid and ethylenediaminetetraacetic acid and polyaspartic acid, polyphosphonic acids, in particular aminotris (methylenephosphonic acid), ethylenediaminetetrakis (methylenephosphonic acid) and 1-hydroxyethane-1,1-diphosphonic acid, polymeric hydroxy compounds such as dextrin and polymeric (poly)carboxylic acids, polymeric acrylic acids, methacrylic acids, maleic acids and mixed polymers thereof, which can also contain polymerized small amounts of polymerizable substances without carboxylic acid functionality. Suitable, although less preferred, compounds of this class are copolymers of acrylic or methacrylic acid with vinyl ethers, such as vinylmethyl ethers, vinyl esters, ethylene, propylene and styrene, in which the acid content is at least about 50% by weight. The organic builder substances can be used, in particular for the preparation of liquid agents, in the form of aqueous solutions, preferably in the form of from about 30 to about 50 percent by weight aqueous solutions. All of the acids mentioned are generally used in the form of their water-soluble salts, in particular their alkali metal salts.

Organic builder substances can, if desired, be included in amounts of up to about 40% by weight, more preferably up to about 25% by weight, and preferably from about 1% to about 8% by weight. Amounts near to the stated upper limit are preferably used in paste-form or liquid, in particular water-containing agents as contemplated herein. Washing aftertreatment agents as contemplated herein, such as, for example, fabric softeners, can optionally also be free of organic builder.

In particular, alkali silicates and, if there are no concerns about their use, also polyphosphates, preferably sodium triphosphate, can be considered suitable water-soluble inorganic builder materials. In particular, crystalline or amorphous alkali metal aluminosilicates can be used as water-insoluble, water-dispersible inorganic builder materials, if desired, in amounts of up to about 50% by weight, preferably not more than about 40% by weight and in liquid agents, in particular from about 1% by weight to about 5% by weight. Preferred among these are the washing agent grade crystalline sodium aluminosilicates, in particular zeolite A, P and optionally X. Amounts near the above upper limit are preferably used in solid, particulate agents. In particular, suitable aluminosilicates have no particles having a particle size greater than about 30 µm and preferably include at least about 80% by weight of particles having a size of less than about 10 µm.

Suitable substitutes or partial substitutes for said aluminosilicate are crystalline alkali silicates which can be present alone or in a mixture with amorphous silicates. The alkali metal silicates useful as builders in the agents as contemplated herein preferably have a molar ratio of alkali metal oxide to SiOz below about 0.95, in particular from about 1:1.1 to about 1:12, and can be present in amorphous or crystalline form. Preferred alkali metal silicates are the sodium silicates, in particular the amorphous sodium silicates, having a molar ratio of NazO:SiOz of from about 1:2 to about 1:2.8. Preferably, crystalline phyllosilicates of the general formula Na₂SiₓO₂ₓ+i - y H₂O are used as crystalline silicates which can be present alone or in a mixture with amorphous silicates, in which x, the so-called modulus, is a number from about 1.9 to about 4 and y is a number from 0 to about 20 and preferred values for x are 2, 3 or 4. Preferred crystalline phyllosilicates are those in which x in the abovementioned general formula assumes the values 2 or 3. In particular, both beta and delta-sodium silicates (Na₂Si₂0s - y H₂O) are preferred. Practically anhydrous crystalline alkali silicates of the abovementioned general formula prepared from amorphous alkali silicates can also be used in agents as contemplated herein, where x in the formula means a number from about 1.9 to about 2.1. In a further preferred embodiment of the agent as contemplated herein, a crystalline sodium layer silicate, as can be prepared from sand and soda, having a modulus of from about 2 to about 3 is used. Crystalline sodium silicates having a modulus in the range from 1.9 to about 3.5 are used in a further preferred embodiment of agents as contemplated herein. If alkali metal aluminosilicate, in particular zeolite, is also present as an additional builder substance, the weight ratio of aluminosilicate to silicate, is preferably from about 1:10 to about 10:1, based in each case on anhydrous active substances. In agents containing both amorphous and crystalline alkali metal silicates, the weight ratio of amorphous alkali metal silicate to crystalline alkali metal silicate is preferably from about 1: 2 to about 2:1 and in particular from about 1:1 to about 2:1.

Builder substances are, if desired, preferably present in the agent as contemplated herein in amounts of up to about 60% by weight, in particular from about 5% by weight to about 40% by weight. Particularly preferred are water-soluble builders in liquid formulations. Washing aftertreatment agents as contemplated hereins, such as fabric softeners, are preferably free of inorganic builder.

Polymeric thickeners in the context of the present disclosure are the polycarboxylates acting as thickening polyelectrolytes, preferably homo- and copolymerisates of acrylic acid, in particular acrylic acid copolymers such as acrylic acid-methacrylic acid copolymers, and the polysaccharides, in particular heteropolysaccharides, and other conventional thickening polymers.

Suitable polysaccharides or heteropolysaccharides are the polysaccharide gums, for example, gum arabic, agar, alginates, carrageenans and their salts, guar, guar gum, tragacanth, gellan, ramzan, dextran or xanthan and their derivatives, for example, propoxylated guar, and also their mixtures. Other polysaccharide thickeners, such as starches or cellulose derivatives, can alternatively or preferably be used in addition to a polysaccharide gum, for example, starches of various origins and starch derivatives, for example, hydroxyethyl starch, starch phosphate esters or starch acetates, or carboxymethylcellulose or its sodium salt, methyl, ethyl, hydroxyethyl, hydroxypropyl, hydroxypropyl methyl or hydroxyethyl methyl cellulose or cellulose acetate.

Acrylic acid polymers suitable as polymeric thickeners are, for example, high molecular weight homopolymers of acrylic acid crosslinked with a polyalkenyl polyether, in particular an allyl ether of sucrose, pentaerythritol or propylene (INCI Carbomer), which are also referred to as carboxyvinyl polymers.

However, particularly suitable polymeric thickeners are the following acrylic acid copolymers: (i) copolymers of two or more monomers from the group of acrylic acid, methacrylic acid and their simple esters, preferably formed with Ci-₄ alkanols (INCI Acrylates Copolymer), which includes, for example, the copolymers of methacrylic acid, butyl acrylate and methyl methacrylate (CAS 25035-69-2) or of butyl acrylate and methyl methacrylate (CAS 25852-37- 3); (ii) crosslinked high molecular weight acrylic acid copolymers, which include, for example, copolymers of C₁₀-₃₀ alkyl acrylates crosslinked with an allyl ether of saccharose or pentaerythritol having one or more monomers from the group of acrylic acid, methacrylic acid and their simple esters, preferably formed with C₁₋₄ alkanols (INCI Acrylates/C 10-30 Alkyl Acrylate Crosspolymer).

The content of polymeric thickener is usually not more than about 8% by weight, preferably between about 0.1 and about 7% by weight, particularly preferably between about 0.5 and about 6% by weight, in particular between about 1 and about 5% by weight and most preferably between about 1.5 and about 4% by weight, for example, between about 2 and about 2.5% by weight, based on the total weight of the cleaning agent.

One or more dicarboxylic acids and/or salts thereof can be added to stabilize the agent as contemplated herein, in particular with high surfactant content, in particular a composition of Na salts of adipic, succinic and glutaric acid, for example, as is available under the trade name Sokalan^{®} DSC. The use is advantageously carried out in amounts of from about 0.1 to about 8% by weight, preferably from about 0.5 to about 7% by weight, in particular from about 1.3 to about 6% by weight and particularly preferably from about 2 to about 4% by weight, based on the total weight of the cleaning agent.

However, if it is possible to dispense with their use, the agent as contemplated herein is preferably free from dicarboxylic acid (salts).

The washing agents as contemplated herein can be compared with reference washing agents to determine the increased anti-pilling performance of the agents as contemplated herein. Such a washing system can be composed as follows (all specifications in weight percent): Reference agent: anionic surfactant from about 5-7%, builder (for example, citric acid and phosphonates) from about 0-1%, caustic soda from about 0-1%, palm kernel fatty acid from about 0-1%, glycerol from about 0-1%, sodium chloride from about 1-3%, boric acid from about 0-1%, further additives (preservative, defoamer, opt. brightener, dye, perfume) 0-1% and balance demineralized water. Agent as contemplated herein: anionic surfactant from about 5-7%, builder (for example, citric acid and phosphonates) from about 0-1%, caustic soda from about 0-1%, palm kernel fatty acid from about 0-1%, glycerol from about 0-1%, sodium chloride from about 1-3%, boric acid from about 0-1%, further additives (preservative, defoamer, opt. brightener, dye, perfume) from about 0-1%, lipase as contemplated herein from about 0.001-0.1 % and remainder demineralized water. The dosage of the liquid washing agent is preferably between about 4.5 and about 6.0 grams per liter wash solution, for example, from about 4.7, about 4.9 or about 5.9 grams per liter wash solution. Preference is given to washing in a pH range between about pH 8 and about pH 10.5, preferably between about pH 8 and about pH 9.

The abovementioned embodiments of the present disclosure comprise all solid, powdery, liquid, gel-like or paste-like administration forms of agents as contemplated herein which, optionally, can also include several phases and can be present in compressed or uncompressed form. The agent can be present as a free-flowing powder, in particular having a bulk density of from about 300 g/l to about 1200 g/l, in particular from about 500 g/l to about 900 g/l or from about 600 g/l to about 850 g/l. The solid administration forms of the agent also include extrudates, granules, tablets or pouches. Alternatively, the agent can also be liquid, gelatinous or pasty, for example, in the form of a non-aqueous liquid washing agent or a non-aqueous paste or in the form of an aqueous liquid washing agent or a water-containing paste. Furthermore, the agent can be present as a one-component system. Such agents include one phase. Alternatively, an agent can also include a plurality of phases. Such an agent is therefore divided into a plurality of components (multicomponent system).

A further object of the present disclosure is a method for the cleaning of textiles, which an agent as contemplated herein is applied in at least one method step.

In various embodiments, the method described above is exemplified in that the agent as contemplated herein is used at a temperature of from about 0-100° C., preferably from about 0-80° C., more preferably from about 30-70° C. and most preferably from about 40-60° C.

These include both manual and mechanical methods, wherein mechanical methods are preferred. Methods for cleaning textiles are generally distinguished by the fact that various cleaning-active substances are applied to the items to be cleaned and washed off after the contact time in a plurality of method steps, or that the items to be cleaned are otherwise treated with a washing agent or a solution or dilution of this agent. All conceivable washing or cleaning methods can be added to in at least one of the method steps for the application of a washing or cleaning agent as contemplated herein and then represent embodiments of the present disclosure. All facts, subjects and embodiments described for agents as contemplated herein are also applicable to this subject as contemplated herein. Therefore, reference is expressly made at this point to the disclosure in the appropriate place with the statement that this disclosure also applies to the above method as contemplated herein.

Since enzymes naturally already have a catalytic activity and they also unfold in media that otherwise have no cleaning power, such as in mere buffer, a single and/or the only step of such a method can be that the only cleaning active component is a lipase being brought into contact with a soiling, preferably in a buffer solution or in water. This represents a further embodiment of this subject of the present disclosure.

Alternative embodiments of this subject matter of the present disclosure are also methods for the treatment of textile raw materials or for textile care, in which an agent as contemplated herein becomes active in at least one method step. Methods for textile raw materials, fibers or textiles having synthetic components are preferred among these.

Furthermore, the present disclosure also encompasses the use of the agent described herein, for example, as washing or cleaning agents as described above, for the (improved) removal of soilings, for example, of textiles.

All facts, subjects and embodiments described for agents as contemplated herein and the lipase are also applicable to the further subjects as contemplated herein. Therefore, reference is expressly made at this point to the disclosure in the appropriate place with the statement that this disclosure also applies to the above method as contemplated herein and the uses as contemplated herein.

### EXAMPLES

### Example 1: Cloning and Expression

Seven metagenome databases and a total of approximately 50,000 clones were screened in an activity-based screening. It was possible to isolate 13 clones which had activity on LB agar plates offset with tributyrin. In addition, the activity of 26 false-positive clones was disproved after repeated inoculation and separation steps. The plasmid DNA could be isolated from the 13 potentially positive clones. The approximate insertion size was determined by employing enzymatic digestion. The isolated plasmid DNA was sequenced sequentially, yielding 10 different open reading frames (ORF). One of the ORF could be associated with a lipase from Pseudomonas stutzeri (PsLip).

The gene was cloned into a pET24a expression plasmid and transformed into the expression strain E. coli BL21 (DE3) pLys for the recombinant expression of the target lipase PsLip. A 1 L culture was grown. The induction was initiated with 0.1 mM IPTG and cultivation was carried out at 37° C. for 24 h. The cells were then disrupted by ultrasound. The crude extract obtained was used for a mini-washing experiment.

### Example 2: Washing Test

A washing test was performed using the crude E. coli extract in which the lipase described was expressed.

Was washed in a common liquid washing agent without enzymes (Table 1) at 40° C. in 16°dH water for 1 h. The enzyme concentration was at 0.32 µg lipase/milliliter of wash solution, which corresponds to a common lipase concentration in washing agents.

TABLE 1

Used washing agent matrix This is a commercial washing agent matrix (without opt. brightener, perfume and dyes) used for the washing test:

| | |
|---|---|
| Chemical name | % weight active ingredient in the formulation |
| Anionic surfactant | 5-7% |
| Nonionic surfactant | 3-5% |
| Builder (citric acid and phosphonates) | 0-1 % |
| Caustic soda | 0-1% |
| Palm kernel oil fatty acid | 0-1% |
| Glycerol | 0-1 % |
| Sodium chloride | 1-3% |
| Boric acid | 0-1% |
| Propylene glycol laurate | - |
| Further additives (preservative, defoamer, opt. brightener, dye, perfume) | 0-1% |
| Water | Radical |

Dosing 4.7 g/L

The following soilings were used in the test:

| | | |
|---|---|---|
| 1.wfk 20D | Pigment/sebum | Polyester/cotton |
| 2.CS 61 | Beef fat, colored | Cotton wool |

The individual fabrics were punched out (diameter = 10 mm) and placed in a microtiter plate. The wash solution, having a final concentration of 4.0 g/L, was preheated to 40° C. Lye and enzyme were then added to the soiling and incubated for 1 h at 40° C. and 600 rpm. After washing, the soiling was rinsed several times with clear water, dried and the brightness determined with a colorimeter.

The lighter the fabric, the better the cleaning performance. The L value = brightness is measured here, the higher the brighter. The brightness values of the test preparations are listed in Table 2.
Sample 1: Washing agent without lipase
Sample 2: Washing agent with PsLip

TABLE 2

Results

| | Sample 1 | Sample 2 |
|---|---|---|
| wfk 20D | 69.19 | 76.89 |
| CS 61 | 74.50 | 78.16 |

It becomes clear that the lipase as contemplated herein shows a good washing performance on all three soilings. A significant improvement in performance is regarded as 1 unit, up to 7.7 units of improvement were achieved here.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A washing or cleaning agent, comprising a lipase having at least about 65% sequence identity with the amino acid sequence specified in SEQ ID NO: 1 over the entire length thereof.

2. The agent according to claim 1, wherein the lipase comprises an amino acid sequence which is identical to the amino acid sequence specified in SEQ ID NO: 1 over its total length to at least 90% .

3. The agent according to claim 1, wherein
(1) the lipase is obtainable from a lipase with the amino acid sequence specified in SEQ ID NO. 1 as a starting molecule by single or multiple conservative amino acid substitution; or
(2) the lipase is obtainable from a lipase with the amino acid sequence specified in SEQ ID NO. 1 as a starting molecule by fragmentation, deletion, insertion or substitution mutagenesis and comprises an amino acid sequence that matches the starting molecule over a length of at least about 182, contiguous amino acids.

4. The agent according to claim 1, wherein the lipase is present in the agent in an amount of from about 0.00001-1% by weight .

5. The agent according to claim 1, wherein
it comprises at least one additional ingredient selected from the group consisting of surfactants, builders, bleaching agents, bleach activators, water-miscible organic solvents, further enzymes, sequestrants, electrolytes, pH regulators, optical brighteners, grayness inhibitors, foam regulators, dyes, and fragrances, .

6. A method for the cleaning of textiles, wherein an agent according to claim 1 is applied to the textiles in at least one method step.

7. (canceled)

8. The method according to claim 5, wherein the agent is present in liquid form.


SEQUENCE LISTING (1 sequence)

SEQ ID NO: 1 - protein, 280 aa, Pseudomonas stutzeri
MRKFTLGCLSATALVALSVSATAAPLPDTPGAPFPSVSSFDNDGPYAVTSQSEGPNCRVYRPRTLGQGGVRHPIILWGNGTGTGPTTYSGLLTHWASHGFVVAAAETSNAGTGREMLACLDYLVQESRTYGTYVGVLNTGRVGTSGHSQGGGGSIMAGQDDRVKATAPIQPYTIGLGHDSSSQRNQRGPMFLMSGGADTIAIPYLNAQPVFTRANVPIFWGERRYVSHFEPVGNGGEYRGPSTAWFRYQLMDDQARSTFYGRLCRLCTSLLWSVERKGIE